(19) United States
(12) Patent Application Publication
(10) Pub. No.: US 2004/0158507 A1
(43) Pub. Date: Aug. 12, 2004

(54) Inventory management and replenishment system

(76) Inventors: Robert B. Meek JR., Pittsburgh, PA (US); Tyler Jones, Dallas, TX (US); Aditya Sethi, Frisco, TX (US); Gregory Hart, Sarver, PA (US); Bridget Ganow, Pittsburgh, PA (US)

Correspondence Address: THORP REED & ARMSTRONG, LLP, ONE OXFORD CENTRE, 301 GRANT STREET, 14TH FLOOR, PITTSBURGH, PA, 15219-1425, US

(21) Appl. No.: 10/730,657

(22) Filed: Dec. 8, 2003

Related U.S. Application Data
(60) Provisional application No. 60/431,293, filed on Dec. 6, 2002.

(51) Int. Cl. G06F 17/60

(52) U.S. Cl. 705/28

(57) ABSTRACT

A method is comprised of various steps, some of which are performed at a healthcare facility and some of which are performed at a distributor's facility. The steps performed at the healthcare facility may include: dispensing medical items from a decentralized storage location; automatically generating data representative of the dispensed items; transmitting the data to a central database; and automatically generating a purchase order from the data. At the distributor's facility, a resupply package containing the items identified in the purchase order is assembled and shipped to the healthcare facility. The resupply package may include items in their original manufacturer packaging. The resupply packages may also be organized so that each package carries items for one or a group of shelves or other storage locations at the healthcare facility. The resupply package may be a tote carrying a bar code that serves as an identifier of the contents of the tote and the location(s) to be replenished. The totes may be used to resupply or replenish a centralized restocking location at the healthcare facility. A system is also disclosed.

The application claims the benefit of U.S. patent application serial No. 60/431,293 filed Dec. 6, 2002 and entitled Inventory Management and Replenishment System, which is hereby incorporated by reference.

### BACKGROUND OF THE INVENTION

1. Field of the Invention

The present invention is directed to inventory management systems and, more particularly, to inventory management systems which enable replenishment of inventory.

2. Description of the Background

Medical facilities, such as hospitals, nursing homes, etc. have a centralized location such as a pharmacy department and/or materials management department within the facility to coordinate the dispensing of drugs and/or medical supplies to the patients of the medical facility. The departments utilizing medications and medical supplies in such facilities have long been burdened with the increasingly complex record keeping and inventory management that results from caring for hundreds, if not thousands, of patients every day. Various methods have been employed to assist a centralized pharmacy or other centralized medical supply departments with maintaining accurate records while attempting to reduce the burden of managing all of the information associated with the distribution of medications and medical supplies. The responsibilities of the centralized supply include: filling individual patient prescriptions on a daily basis; administration of drugs using the five rights: right drug, right patient, right dose, right time and right route; dispensing medical supplies to patients; maintaining sufficient inventory of each drug or medical supply so as to have sufficient quantities on hand to administer to patients on a daily basis; tracking of drug interactions to prevent a patient from being given a drug that has adverse affects when combined with other drugs; accounting for the purchase of medications and medical supplies for use in the facility; accounting associated with dispensing of medications and medical supplies to individual patients; tracking of medication expiration dates to rid inventories of expired medications; and tracking of drug lot numbers, for example, in the event of a recall of a particular drug or drug lot number.

Medical facilities will dispense medications in one of three modes: centralized, decentralized, or a hybrid of partial decentralization. In facilities that are partly or fully decentralized, a very important function of the centralized pharmacy or materials management department is to restock various inventory locations, e.g. nurses stations, unit-based cabinets, satellite pharmacies, or off-site facilities in a network, with the quantity and types of medications and medical supplies that must be dispensed by the decentralized locations on a daily basis.

The need for storage locations in medical facilities remote from the centralized storage location stems from the need to be able to quickly and conveniently dispense medications and medical supplies (whether controlled or uncontrolled) to patients. To be able to dispense, there must be adequate supplies of the medications and medical supplies in the remote storage locations. The contents of these remote storage locations varies depending upon the medical procedures practiced in the area where the storage location is situated. For example, a storage location near an emergency room will be stocked differently than a storage location next to a surgical suite. Thus, to maintain the proper level of medications and medical supplies, accurate inventory control is necessary.

Pharmacy departments that have embraced the decentralized distribution paradigm face the challenges of tracking, optimizing and replenishing inventory associated with the decentralized storage locations. Pharmacy departments have multiple systems which are not connected resulting in: data that is not synchronized between systems; the inability to accurately value their inventory; and the inability to accurately report on what is dispensed through centralized and decentralized technology.

Replenishing the inventory in a centralized hospital-wide nursing unit-based cabinet (UBC) system is a time consuming, laborious, and often awkward process for pharmacy departments. The typical manual pick process using paper-based replenishment reports is certainly not optimized. Thus, pharmacy departments are very interested in a better way of managing their current UBC replenishment processes.

Keeping a large number of UBC's stocked with optimal inventory levels based on utilization is another big challenge for a pharmacy department. Over time, the hospital's UBC inventory “supply and demand” equation goes out of tune resulting in frequent stock-outs with multiple unscheduled UBC replenishment cycles per day and inventory becoming stale due to overstock in the cabinets. The result is costly waste due to inventory carrying cost, expired meds, general obsolescence, and the opportunity cost of misappropriating cabinet space. In turn, that results in a frustrated pharmacy staff and an even more disappointed nursing department. Ultimately patient care could be compromised by lack of the needed drug (UBC stock out) or if the drug in the cabinet is expired and no longer usable. Pharmacy departments are unable to “re-tune” their UBC's on their own. Manually analyzing dispensing records, trends, and stock-out incidents is daunting. Nearly all pharmacy departments do not have the technological means or available analysts to accomplish this level of work on their own. The result is cabinets that grow more out-of-tune with each passing day. Thus, the need exists for an automated method for calculating a distributor order and an efficient method for restocking the centralized location with a received distributor order.

### SUMMARY

One aspect of the present disclosure is a system comprising a plurality of decentralized locations each containing dispensing hardware (e.g., a plurality of dispensing cabinets) for dispensing items (medication or supplies) and for generating data representative of the dispensed items. That data is sent to a central database which generates restocking orders. A restocking order is, broadly speaking, simply a grouping of items needed to restock or replace the items that have been dispensed from the decentralized location. The restocking orders are received at a central restocking location where a restock package is prepared from various types of restocking hardware (e.g., carousels, open shelving, narcotics vault, etc.). Based on either the items placed into the restocking packages or on the dispensing data, the central database identifies those items that must be replenished or resupplied by reordering those items from a distributor, supplier, vendor, or other source of such items. Those items that must be replenished or resupplied are identified in a purchase order that is automatically generated. The purchase order may be subject to a manual review if desired. An online data exchange system is responsive to the purchase orders for communicating the purchase orders to a distributor or other source of the items. At the distributor's facility, a resupply package containing the items identified in the purchase order is assembled and shipped to the healthcare facility. The resupply package may include items in their original manufacturer packaging. The resupply packages may also be organized so that each package carries items for one or a group of shelves or other storage locations at the central restocking location. The resupply package may be a tote carrying a bar code that serves as an identifier of the contents of the tote. The totes may be used to resupply or replenish the centralized restocking location at the healthcare facility.

Another aspect of the present disclosure is a method comprised of various steps, some of which are performed at a healthcare facility and some of which are performed at a distributor's facility. The steps performed at the healthcare facility may include: dispensing medical items from a decentralized storage location; automatically generating data representative of the dispensed items; transmitting the data to a central database; and automatically generating a purchase order from the data. At the distributor's facility, a resupply package containing the items identified in the purchase order is assembled and shipped to the healthcare facility. The resupply package may include items in their original manufacturer packaging. The resupply packages may also be organized so that each package carries items for one or a group of shelves or other storage locations at the healthcare facility. The resupply package may be a tote carrying a bar code that serves as an identifier of the contents of the tote. The totes may be used to resupply or replenish a centralized restocking location at the healthcare facility.

Another aspect of the present disclosure is a method for dispensing, restocking, reordering and replenishing medical items in a healthcare facility. Steps performed at the healthcare facility may include: dispensing medical items from a decentralized location; automatically generating data representative of the dispensed items; transmitting the data to a central database; automatically generating restocking orders from the data; generating a restocking package at a central restocking location from the restocking order; restocking the decentralized storage location with the restocking package; automatically assembling a purchase order from the data; and transmitting the purchase order to a distributor. At the distributor's facility, a resupply package containing the items identified in the purchase order is assembled and shipped to the healthcare facility. The resupply package may include items in their original manufacturer packaging. The resupply packages may also be organized so that each package carries items for one or a group of shelves or other storage locations at the healthcare facility. The resupply package may be a tote carrying a bar code that serves as an identifier of the contents of the tote. The totes may be used to resupply or replenish the central restocking location at the healthcare facility.

The present disclosure enables hospital pharmacies to gain control over managing their decentralized pharmacy model. The present disclosure builds in control, quality, and discipline in a customer's daily unit based cabinet replenishment process. Optimal inventory management is assured thereby taking the guess work out of the daily restocking and replenishment processes. Hospitals maintain control over purchasing and maximize contract compliance. With the present disclosure, pharmacy technician labor freed can be redeployed to other more valuable tasks (such as inventory management and purchase trend analysis and reporting). Satisfaction of the nursing staff with the pharmacy department is improved due to far fewer cabinet stock-outs (i.e. inventory is not available). Patient safety will not be compromised by the distraction of making impromptu calls to pharmacy to request a re-stock. Satisfaction of the hospital pharmacy staff is improved due to a more rigorous, solid process for managing, restocking and replenishing cabinet inventory and far fewer unscheduled trips to cabinets for “emergency” stock-out situations. Those, and other advantages and benefits will become apparent from the Detailed Description herein below.

### BRIEF DESCRIPTION OF THE DRAWINGS

For the present invention to be easily understood and readily practiced, embodiments of the present invention will now be described, for purposes of illustration and not limitation, in conjunction with the following figures, wherein:

FIG. 1 is a diagram illustrating the relationship between a centralized storage location and, among other things, a plurality of storage locations;

FIG. 2 is a diagram illustrating a process for distributing items and restocking of items based, at least in part, on records created during distribution;

FIG. 3 is one example of hardware located at a decentralized location implementing a closed system for performing dispensing operations;

FIG. 4 is one example of hardware located at the central location for enabling the manual assembly of restocking packages based on data generated by the hardware of FIG. 3;

FIG. 5 is a diagram illustrating the flow of information between computers used at various locations within a dispensing/restocking system;

FIG. 6 is a block diagram of the system of the present invention;

FIG. 7 illustrates one example of manufacturer packaging; and

FIG. 8 is a flow chart illustrating the steps of a software application for analyzing and adjusting inventory.

### DETAILED DESCRIPTION

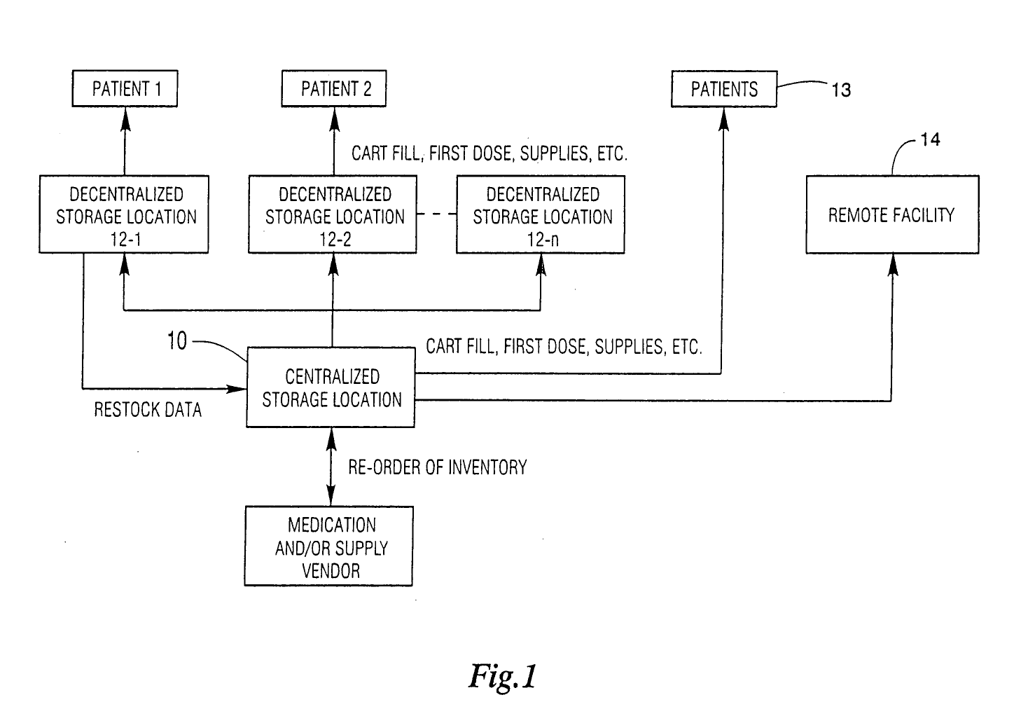

FIG. 1 is a diagram illustrating the relationship between a centralized storage location 10 and various inventory destinations, including a plurality of decentralized storage locations 12-1, 12-2 through 12-n, patients 13, and a remote facility 14. Each of the decentralized storage locations 12-1 through 12-n is capable of dispensing items stored at the location. The items may include medications, controlled medical supplies, medical supplies or items of a nature consistent with the facility in which the system illustrated in FIG. 1 is located. Items may be dispensed directly from centralized storage location 10 to patients 13, or from the centralized storage location 10 to a remote facility 14. Data typically flows from the decentralized storage locations 12-1 through 12-n to the centralized storage location 10. In response to that data, items are typically moved from the central storage location 10 to the decentralized storage locations 12-1 through 12-n or to the remote facility 14 to restock such locations to either replenish dispensed items or to stock new items. Decentralized locations could include satellite pharmacies, computerized medication cabinets, stationary/mobile medication carts, nurse servers, remote hospital pharmacies, supply closets, supply cabinets, etc. Supplies can be reordered from distributors based on levels of stock in the centralized storage location 10.

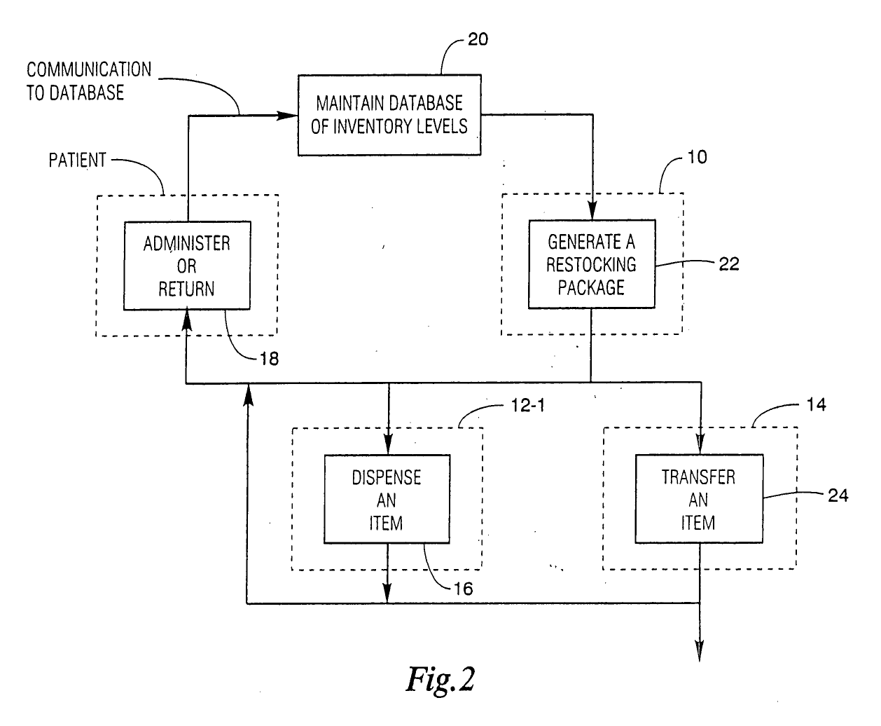

FIG. 2 illustrates a process which may begin with a step of dispensing an item at step of 16 from one of the decentralized storage locations 12-1 to a patient. A dispensing operation may occur in a variety of ways. In a medical facility, dispenses may be completed from medication orders or they may be completed from inventory lists, to name a few types of dispensing operations. Assuming a medication has been dispensed from decentralized storage location 12-1, the medication may either be administered to a patient or returned as shown by step 18. Medications may be returned for a variety of reasons such as the patient has checked out, been moved, or the patient's medication may have been changed. Medications may be returned to the decentralized storage location 12-1. Certain types of medications may simply be replaced in the decentralized storage location 12-1 so as to be used in another dispensing operation, or may need to be disposed of.

The administration of medications occurring at step 18 may be carried out through the use of a hand-held device such as an AcuScan-Rx™ device available from McKesson Automation Inc., 700 Waterfront Drive, Pittsburgh, Pa. Such devices are wireless devices which communicate with a database to verify the administration of medications to patients. Such communications enable the maintenance of a database of inventory levels as shown by step 20. The database and associated computer system for maintaining the database of inventory levels may be located at the centralized storage location 10 or may be located remote therefrom. In either event, the computer system necessary for maintaining the database provides information which enables the centralized storage location 10 to perform step 22 of generating a restocking package. The generation of the restocking package may be done completely automatically, manually, or through some combination of manual and automatic processes. The restocking package is used to restock the decentralized storage location 12-1.

Restocking packages may also be generated at centralized location 10 and delivered to the remote facility 14. From facility 14 an item may be transferred as shown by step 24. The transfer may be a dispensing step for a patient or a transfer to another location. Items may also be dispensed directly to the patient from the centralized location 10.

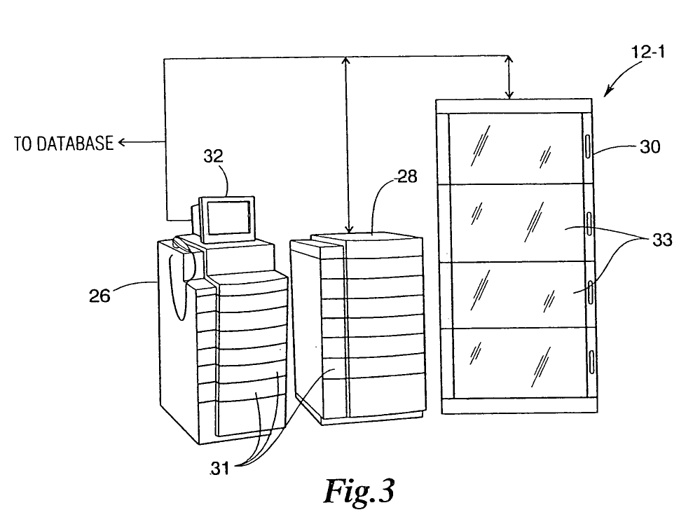

FIG. 3 illustrates one example of hardware which may be located at any of the decentralized locations 12-1 through 12-n. The hardware illustrated in FIG. 3 is comprised of an AcuDose-Rx™ cabinet 26, having a control computer 32, and an AcuDose-Rx™ auxiliary cabinet 28, available from McKesson Automation Inc. A supply tower 30 is also illustrated. The control computer 32 controls the operation of the cabinet 26, auxiliary cabinet 28, and supply tower 30. The control computer 32 is also in communication with the central database. The reader will understand that the hardware illustrated in FIG. 3 is exemplary and is illustrated for purposes of demonstrating one type of hardware which may be located at the decentralized storage locations 12-1 through 12-n.

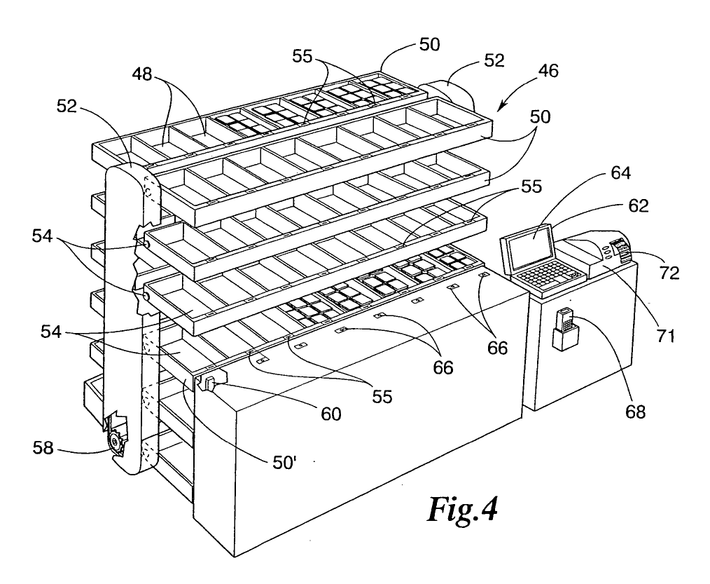

FIG. 4 illustrates one example of hardware located at the central location 10 for enabling the manual assembly of a restocking package based on data generated by the hardware illustrated in FIG. 3. FIG. 4 illustrates a carousel 46 which may be of the type disclosed in U.S. application Ser. No. 09/998,488, filed on Nov. 30, 2001 and entitled Carousel Product For Use In Integrated Restocking And Dispensing System, the entirety of which is hereby incorporated by reference. The carousel 46 is comprised of a plurality of bins 48 arranged in a plurality of rows 50. The rows 50 of bins 48 are connected to a drive track 52, which may be, for example, a pair of endless belts or chains. The rows 50 of bins 48 are connected to the drive track 52 through a swivel connection 54 which enables the rows 50 of bins 48 to maintain a horizontal position as the rows 50 are driven by the drive track 52. Each of the bins 48 carries indicia 55, which may be, for example, a barcode and/or a label indicating the contents of the bin.

The drive track 52 is driven by, for example, an electric motor 56. The electric motor 56 may drive the drive track through one or more drive gears 58 in the case of a chain type of drive track or through a pulley in the case of a belt type of drive track. In addition to use of an electric motor 56, hydraulics or any other appropriate mechanism for driving the drive track 52 may be used. A sensor 60 may be provided to sense the position of the rows 50 of bins 48. Alternatively, a shaft encoder may be provided for motor 56 for keeping track of the degree of rotation of the motor's 56 shaft and, through knowledge of the gearing and the previous position of the rows 50, the position of the rows can be controlled. Use of the word “sensing” is intended to cover any of the various known method of sensing and/or calculating the position of the rows 50.

The carousel 46 is under the control of a workstation 62, which may be comprised of a personal computer in communication with the database. The workstation 62 receives information from the database regarding items, and quantities for each item, needed to replenish each of the decentralized storage locations 12-1 through 12-n or to fulfill patient dispenses. The workstation 62 processes the information and presents to the user through a screen 64 a series of operations referred to as “picks”. The information displayed on the screen may include, for example, an identification of the decentralized storage location, an identification of a cabinet, tower, shelving unit, etc. at the decentralized location, an identification of the patient, the item and quantity to be picked. The workstation 62 also controls a printer 71 which can print barcode labels 72.

A label 72 with a barcode indicating the item (medication, supply, or kit) and the destination (cabinet, patient, etc.) will be printed from the printer 71, the user will scan that barcode with a scanner to activate the carousel picking process. The carousel dynamically evaluates the work queue of requests (patient dispenses, cabinet refills, on demand picks, stat, now, etc.) based on a configured set of priorities, set by the user. These priorities allow a medical facility to configure the order in which the different requests will be processed. Additionally, the facility may set up different priority ordering for different time periods in the day. For example, first doses may be disabled or prioritized lower during the hours of a cart fill. This work queue can also be paused at any time to perform an on-demand pick or restock. That allows user to pick an urgent item that may be in the queue.

To enable a pick to be performed, the workstation 62 activates motor 56 to bring the row 50 having the desired item into a pick position. In FIG. 4, the row 50′ is illustrated in the pick position. When in the pick position, a plurality of indicia 66 are adjacent to each of the bins 48 in the row 50′ in the pick position The indicia may include, for example, LEDs or an alphanumeric display. The location could also be indicated on an LCD Display or workstation 62. The workstation 62 may cause a number of LEDs equal to the quantity of items to be picked and adjacent to the bin 48 having the items to be picked to illuminate. Alternatively, an alphanumeric screen could be lit with the quantity of items to be picked from the adjacent bin 48. That is sometimes referred to as “pick-to-light” technology.

To finish the pick, a hand-held wireless device 68 is used to scan the bin label or item barcode 55. If the quantity that was picked from the device was not the full requested amount (because of an out of stock or expiration condition), the user can adjust the quantity picked and record a reason for the discrepancy before completing the pick. The barcode 72 that is printed at the beginning of the process (which initiates the picking) and the barcode on the bin or item that is scanned to complete the process are different formats to require the user to scan each of these barcodes (if the same information was encoded in each barcode, the user could scan one of the barcodes twice and would lose a critical validation to prevent picking of the incorrect item). An alternative method of indicating the completion of the pick could be to push a button or any other physical manifestation intended to represent the completion of the pick. The user then moves to the workstation 62 and initiates the next pick.

Other types of hardware which may be used at the centralized storage location 10 include a system of the type disclosed in U.S. Pat. No. 5,593,267 entitled “Automated System for Selecting and Delivering Packages from a Storage Area,” U.S. Pat. No. 5,880,443 entitled “Automated System for Selecting Packages from a Cylindrical Storage Area,” and U.S. patent application Ser. No. 09/480,819 entitled “An Automated Medication Dispensing System,” all of which are hereby incorporated by reference. It is anticipated that the centralized location may be comprised of various types of hardware such as the carousel illustrated in FIG. 4 and/or the hardware identified in the aforementioned patents and pending application. The centralized storage location may be completely automated, partially automated by having both a carousel and, for example, a computer-controlled robot, or completely manual by having one or more carousels. In that manner, a manual restocking system based on a carousel can be used side-by-side with an automated restocking system based on a robot.

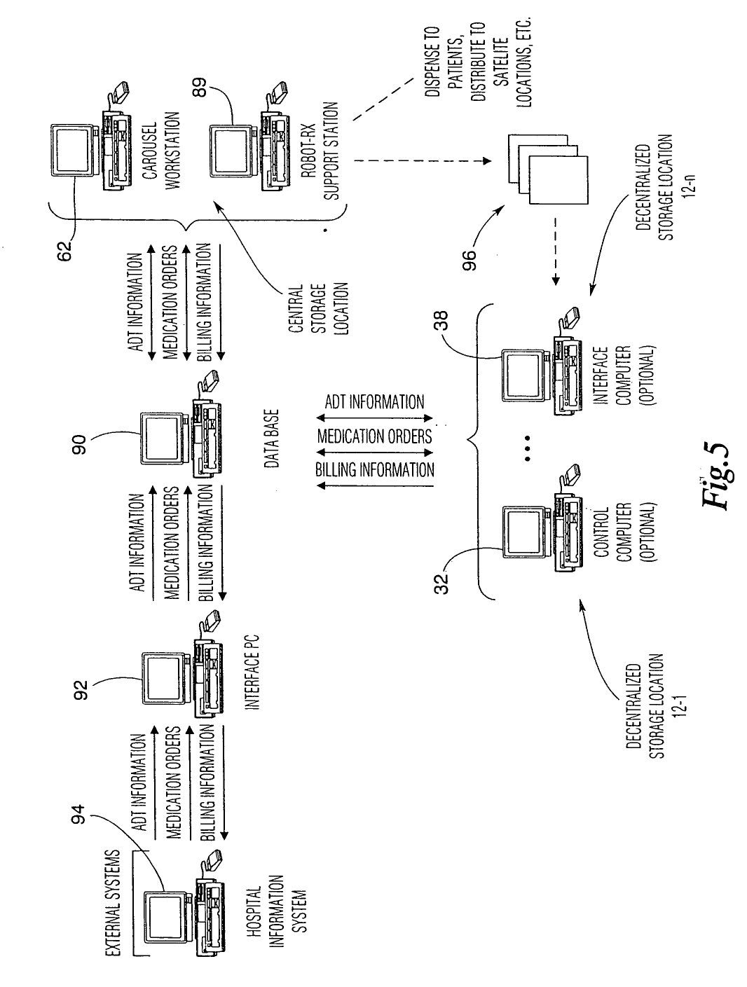

FIG. 5 illustrates the computers used at various locations within a dispensing/restocking system of the type disclosed herein. As seen in FIG. 10, decentralized storage location 12-1 is where control computer 32 (if supplied) is located. Decentralized storage location 12-n is where an interface computer 38 (if supplied) is located. The carousel work station 62 is located at the centralized storage location 10. The centralized storage location 10 may also have a Robot-Rx support station 89 which is used to control a robot.

A computer 90, which may be located at centralized storage location 10 or may be located elsewhere, maintains the database for the system. The computer 90 receives information from the decentralized storage locations 12-1 through 12-n and provides information to the carousel work station 62 and/or the Robot-Rx support station 89 to enable restocking packages 96 to be prepared. Additionally, dispenses to patients, distributions to satellite facilities, and the like may occur from centralized location 10. An interface PC 92 may be provided to enable external systems, such as a PC 94 on which a hospital information system resides, to communicate with the computer 90 on which the database is located. Completing the description of FIG. 5, as has been previously described, restocking packages 96 are prepared at the centralized storage location 10 and delivered to the decentralized storage locations 12-1 through 12-n. As a result of the creation of the restocking packages 96, the carousel 46 or other automation device needs replenished or refilled.

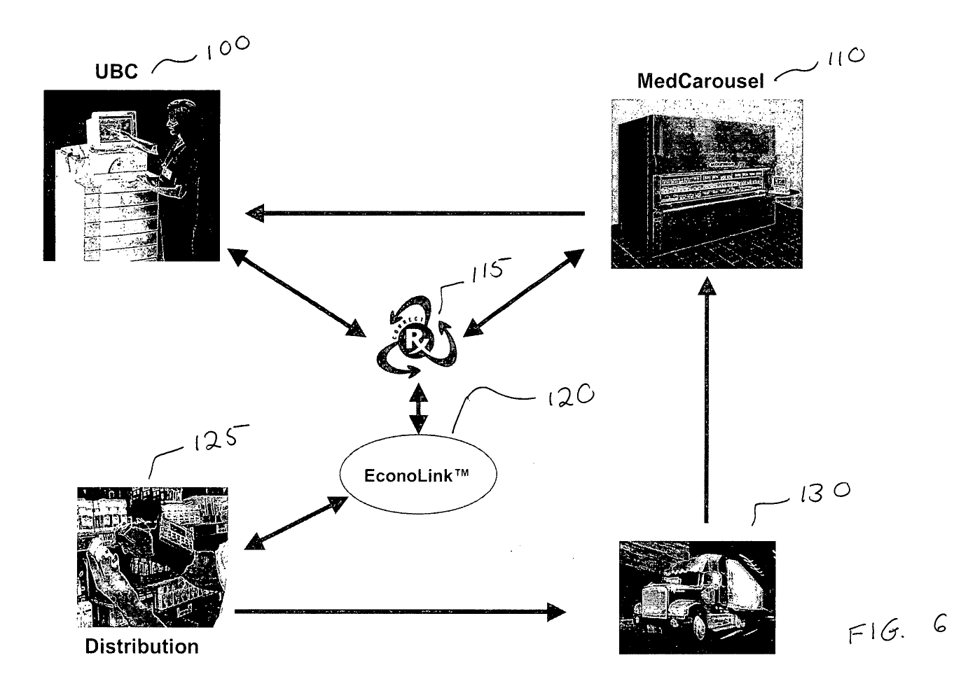

One embodiment of the system of the present invention is illustrated in FIG. 6 and may be comprised of four (4) elements:

1. one or more decentralized, unit-based cabinet (UBC, e.g. AcuDose-Rx cabinets 26) or other type of dispensing/inventory management equipment or system 100;

2. a centralized restocking location 110 which may contain automation equipment (e.g. ROBOT-Rx robot, MedCarousel carousel, NarcStation narcotics vault, etc. ) or manual devices (open shelving), or a combination thereof, from which packages are assembled for restocking the dispensing/inventory management equipment or system(s) 100;

3. a centralized inventory management system 115 which may be comprised of, for example, Connect-Rx software and database available from McKesson Automation, Inc.; and

4. an electronic inventory ordering system 120 such as, for example, McKesson's Econolink2000 and Supply Management Online (SMO) systems.

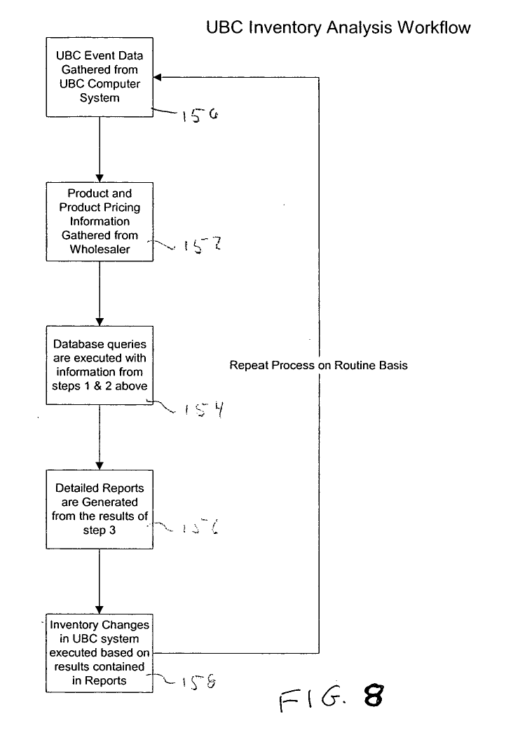

The system may also contain an optional software application (see FIG. 8 discussed below) designed to analyze the inventory contained in a unit-based cabinet system, and to provide reports that pharmacy personnel may use to make adjustments and changes to the inventory contained in the unit-based cabinet system.

In one embodiment, a hospital maintains medication inventory in unit-based cabinet systems 100 installed throughout the hospital. The UBC systems maintain perpetual inventory records for the inventory contained in each cabinet 26 and transmits those records to the centralized inventory management system 115. That may be performed either by each cabinet 26 or by a control computer operating one or more cabinets. When items contained in a cabinet fall below certain trigger levels, those items are identified either by the individual cabinet 26 or the control computer to the centralized inventory management system 115 as items requiring restocking in the UBC's.

At certain times during the day, pharmacy personnel operating in the restocking location 110 are required to restock the inventory in the UBC's using, for example, devices such as the carousel 46 and/or NarcStation narcotics vault. The centralized inventory management system 115 processes the list of items that require restocking in the UBC's and presents the correct list (e.g. a restocking report) of medications/supplies to pharmacy personnel for withdrawal from the centralized inventory at restocking location 110. After completion of this activity, the selected inventory is then transported to the dispensing devices (e.g., UBC's) for restocking. This technology eliminates the need to run hard-copy refill reports for unit-based cabinet restocking, allows cabinets to be grouped into “delivery units” for efficient medication retrieval and distribution, enables the carousel 46 to automatically determine the most efficient picking path and present shelves to the pharmacy technician thereby lowering the pharmacy labor typically utilized for dispensing device restocking activities.

Similar to the UBC system 100, centralized inventory management system 115 maintains perpetual inventory records for the inventory maintained in the restocking location 110. This inventory may be physically contained within the automation devices (e.g. carousel, narcotics vault, etc.), or may simply be tracked by the centralized inventory management system 115. When items contained in the restocking location fall below certain trigger levels, those items are identified in the centralized inventory management system 115 as items requiring replenishment from an outside supplier or vendor such as McKesson Health Systems.

At a certain time or times during the day, pharmacy personnel operating in the restocking location 110 are required to replenish items maintained in the restocking location 110. Utilizing functionality contained in the centralized inventory management system 115, items that require replenishment from an outside supplier or distributor are identified either directly from the data representative of the dispensed items or indirectly from that same data via the restocking reports. The data representative of those items requiring replenishment is automatically assembled electronically into a purchase order. The purchase order can be reviewed and manually modified if desired. The items on the purchase order, including those items modified as a result of the review, are transmitted electronically to an outside supplier's or distributor's procurement system using a system such as McKesson Health Systems' Econolink2000 or Supply Management Online (SMO) systems 120.

The Econolink2000/Connect-Rx Interface is a two-way, online data exchange technology that provides a connection, via the Connect-Rx software, between McKesson Automation products and McKesson Health Systems distribution centers. The technology provides “one-click” order placement for medications managed by all McKesson Automation Products (ROBOT-Rx robot, MedCarousel carousel, AcuDose-Rx dispensing cabinet, NarcStation narcotics vault, etc.). A simple start-up synchronization routine updates Connect-Rx formulary with Econlink 2000 or Supply Management Online proprietary numbers, product package sizes and product acquisition cost at the NDC level. An automated daily maintenance routine provides regular data updates. The technology also simplifies the receipt verification process for items delivered from McKesson Health Systems

For items being replenished in a carousel, the centralized inventory management system 115 may create a location specific order, e.g. an order at the shelf level, grouping all items contained on one or more carousel shelves or rows into one order. The customer may be given the flexibility to determine the number and type of locations to which each order is responsive depending on the location and type of restocking hardware. Upon receipt of a customer's purchase order, the outside supplier prepares at step 125 and then ships at step 130 the ordered inventory items. Preparation includes placing the ordered items (contained in the manufacturer's original packaging) into bar coded totes that are used to ship the inventory to the customer. A message generated by the outside supplier's computer is also sent to the customer. The message may link the tote's bar code to the contents of the tote and the location to be replenished, among others. Upon receipt of the bar coded totes, the customer may scan the bar code affixed to the tote to automate the process of inventory replenishment.

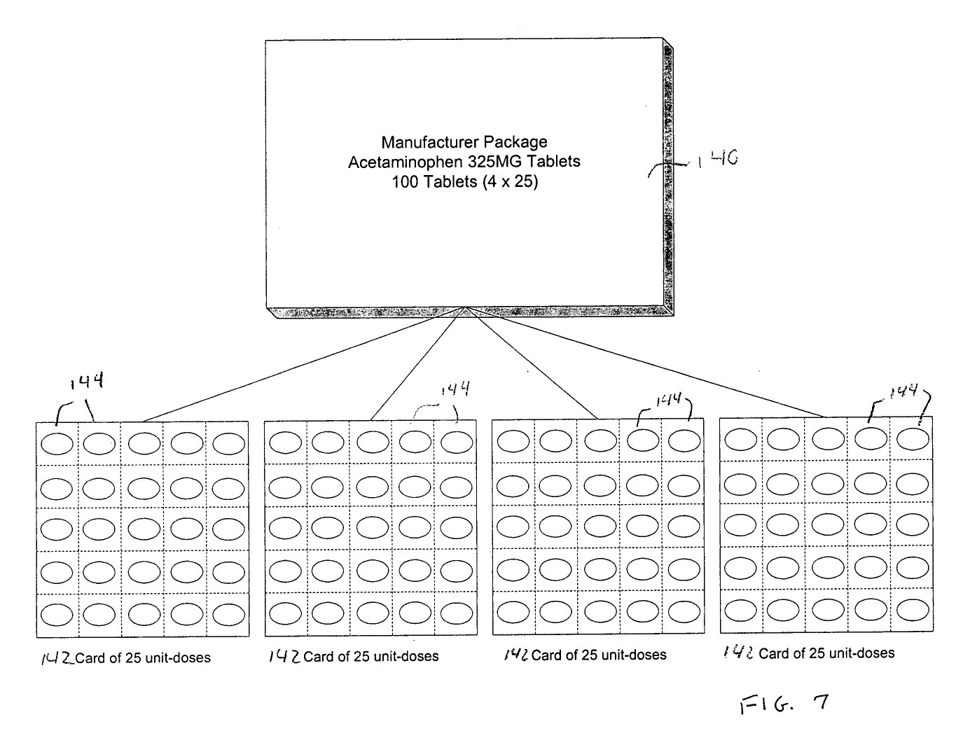

FIG. 7 illustrates one example of manufacturer packaging. In FIG. 7 a manufacturer package 140 contains four cards 142, with each card 142 containing twenty-five individual or unit doses 144 of medication. The entire package 140 would be shipped in a tote; the breakdown of the package 140 into unit doses 144 occurs at the customer location. That enables the distribution center to operate in a very efficient manner.

Customers may also receive a routine analysis of the inventory levels contained in their UBC system. This analysis provides a series of reports that may be used by the customer to make adjustments and modifications to the existing inventory in the UBC system with the goals of achieving optimal medication stock levels, and reducing overall inventory costs. This service analyzes medication usage across the unit-based cabinet system, recommends proper inventory stocking levels based on medication usage over a defined period of time and provides cost savings associated with proper inventory management. One example of the steps of such an analysis is shown in FIG. 8.

Turning to FIG. 8, at 150 event data representative of dispensing events is gathered. At 152, product and pricing information is gathered. Using that data, one or more queries may be executed at 154 with the results used to generate one or more reports at 156. Based on the reports, inventory changes may be executed at 158. The process may be repeated periodically.

Hospitals face numerous operational challenges, such as labor, staffing, patient safety, quality assurance, and workflow management. To help address these challenges, the present disclosure integrates ordering and inventory management software with automation technologies into one unique solution. With its streamlined functionality, the present disclosure empowers hospitals to optimize both pharmacy operations and asset management.

The present disclosure provides for automated, streamlined cabinet restocking including: efficient inventory tracking; optimization of cabinets based on usage analysis; compatibility with any unit-based cabinet, regardless of manufacturer; and true low-unit-of-measure distribution to the hospital's cabinet network. The present disclosure further provides automated, electronic re-ordering including automatic calculation of suggested order quantities based on usage and the option to place orders by product and DEA classification.

The present disclosure also provides bar code driven replenishment including: daily order delivered in location specific (e.g. shelf-specific) totes; the totes are scanned and instantly identified at the restocking hardware for efficient restocking at the location (e.g. shelf) level; and “closed loop” inventory management for UBC cabinet systems.

The present disclosure provides inventory control including “virtual inventory” departments created for management of all inventory locations throughout the hospital. Cabinet stock-outs are dramatically reduced to ensure nursing access to needed medications. The present disclosure also provides real-time inventory valuation including: automated daily updates of dose-level acquisition prices; NDC, Econlink 2000 and Supply Management Online proprietary numbers and manufacturer packaging sizes; easy generation of inventory valuation reports based on current contract pricing; and rapid access to inventory management and trending reports.

While the present invention has been described in connection with exemplary embodiments thereof, those of ordinary skill in the art will recognize that many modifications and variations are possible. Accordingly, the scope of the present invention is intended to be limited only by the following claims and equivalents thereof.

What is claimed is:

1. A method, comprising:
at a health care facility:
generating data representative of dispensed items;
transmitting said data to a central database;
automatically assembling a purchase order from said data;
transmitting the purchase order to a distributor; and
at a distributor location:
assembling the items, in each item's manufacturer packaging, identified in said purchase order; and
shipping the assembled items to the healthcare facility.

2. The method of claim 1 additionally comprising, before transmitting said purchase order, manually reviewing said purchase order.

3. The method of claim 1 additionally comprising using the shipped items to replenish a restocking location at the health care facility.

4. The method of claim 1 wherein said assembled items are shipped in a tote, said method additionally comprising marking said tote with a bar code.

5. The method of claim 4 additionally comprising notifying the healthcare facility of the bar code and identifying the items in the tote associated with said bar code.

6. A method, comprising:
generating data representative of items dispensed in a healthcare facility;
transmitting said data to a central database;
automatically assembling one or more purchase orders from said data, wherein each said purchase order represents items from one or more locations within in a restocking location; and
transmitting the purchase order to a distributor.

7. The method of claim 6 additionally comprising, before transmitting said purchase order, manually reviewing said purchase order.

8. The method of claim 6 additionally comprising:
at a distributor location:
assembling the items identified in said purchase order; and
shipping the assembled items to the healthcare facility.

9. The method of claim 8 additionally comprising using the shipped items to replenish said one or more locations within the restocking location at the healthcare facility.

10. The method of claim 9 wherein said assembled items are shipped in a tote, said method additionally comprising marking said tote with a bar code.

11. The method of claim 10 additionally comprising notifying the healthcare facility of the bar code, and identifying the items in the tote associated with said bar code and said one or more locations.

12. A method, comprising:
at a health care facility:
dispensing medical items from a decentralized storage location;
automatically generating data representative of the dispensed items;
transmitting said data to a central database;
automatically generating a restocking order from said data;
generating a restocking package at a restocking location from said restocking order;
restocking said decentralized storage location with said restocking package;
automatically assembling a purchase order from said data;
transmitting the purchase order to a distributor; and
at a distributor location:
assembling the items, in each item's manufacturer packaging, identified in said purchase order; and
shipping the assembled items to the healthcare facility; and
at said healthcare facility, replenishing said restocking location with said shipped items.

13. The method of claim 12 additionally comprising, before transmitting said purchase order, manually reviewing said purchase order.

14. The method of claim 12 wherein said assembled items are shipped in a tote, said method additionally comprising marking said tote with a bar code.

15. The method of claim 14 additionally comprising notifying the healthcare facility of the bar code and identifying the items in the tote associated with said bar code.

16. A method, comprising:
at a health care facility:
dispensing medical items from a decentralized storage location;
automatically generating data representative of the dispensed items;
transmitting said data to a central database;
automatically generating a restocking order from said data;
generating a restocking package at a restocking location from said restocking order;
restocking said decentralized storage location with said restocking package;
automatically assembling one or more purchase orders from said data,
wherein each said purchase order represents items from one or more locations within a restocking location;
transmitting the purchase order to a distributor; and
at a distributor location:
assembling the items identified in said purchase order; and
shipping the assembled items to the healthcare facility; and
at said healthcare facility, replenishing said restocking location with said shipped items.

17. The method of claim 16 additionally comprising, before transmitting said purchase order, manually reviewing said purchase order.

18. The method of claim 16 wherein said assembled items are shipped in a tote, said method additionally comprising marking said tote with a bar code.

19. The method of claim 18 additionally comprising notifying the healthcare facility of the bar code and identifying the items in the tote associated with said bar code and said one or more locations to be replenished.

20. A system for dispensing, restocking and reordering medical items in a healthcare facility, comprising:
a plurality of decentralized locations, each containing dispensing hardware for dispensing items and for generating data representative of the dispensed items;
a central database for receiving said data, said central database generating restocking orders for restocking each decentralized location and for generating purchase orders in response to said data;
restocking hardware, responsive to said restocking orders, said restocking hardware having a plurality of storage locations; and
an online data exchange system responsive to said purchase orders for communicating said purchase orders to a distributor, wherein each said purchase order is responsive to items needed to replenish one or more of said plurality of storage locations.

21. The system of claim 20 wherein said restocking hardware includes a carousel and said plurality of storage locations correspond to shelves of said carousel.

22. The system of claim 20 additionally comprising a bar coded tote for carrying items identified in a purchase order.

23. The system of claim 22 additionally comprising a distributor computer for notifying the healthcare facility of the bar code and for identifying the items in the tote carrying said bar code and said one or more storage locations to be replenished.

24. The system of claim 22 wherein items in the tote are in their original manufacturer packaging.
